(19) United States
(12) Patent Application Publication
(10) Pub. No.: US 2016/0095789 A1
(43) Pub. Date: Apr. 7, 2016

(54) Devices and Methods for Treatment and Promotion of Healing of Injured Sites

(71) Applicant: MMJ Labs LLC, Atlanta, GA (US)

(72) Inventors: Amy L. Baxter, Atlanta, GA (US); Louis A. Calderon, Atlanta, GA (US)

(21) Appl. No.: 14/874,909

(22) Filed: Oct. 5, 2015

Related U.S. Application Data
(60) Provisional application No. 62/059,754, filed on Oct. 3, 2014.

(51) Int. Cl. A61H 23/00; A61F 7/00

(52) U.S. Cl. CPC A61H 23/00; A61F 7/00

(57) ABSTRACT

Disclosed herein is a device comprising a casing; at least one vibratory source; at least one power source; elements to actuate the vibratory source; electrical communication between the vibratory source, the power source, and other elements; and an optional thermal element for modulating the temperature of a contacted area of a surface. Also disclosed herein are methods for using the device.

### RELATED APPLICATIONS

This application claims the benefit and priority of U.S. Provisional Patent Application No. 62/059,754, filed Oct. 3, 2014, which is herein incorporated in its entirety.

### TECHNICAL FIELD

The present invention generally relates to devices and methods for the improvement of healing of injured sites in a subject, for example, musculoskeletal injuries or chronic wounds, using devices capable of vibration, with or without thermal effects.

### BACKGROUND

Injured sites in a subject comprise acute or chronic conditions in which normal cellular arrangements or metabolism are disturbed. Such injured sites are detrimental to the subject, for example in creating pain or disability, and are also a major healthcare cost. For example, lower back pain and injury healing represent significant healthcare problems. Chronic wounds are a source of cost and disability, especially in diabetic subjects. Approximately 25% of the 150 million diabetics worldwide will experience a chronic wound, and the majority of these wounds will remain unhealed after 20 weeks of treatment. Normal wound healing comprises overlapping periods of inflammation, angiogenesis, tissue remodeling and tissue formation. Inflammation can decrease and delay healing and increase pain. For injuries, common treatment is ice and elevation to reduce inflammation, but cold alone can diminish angiogenesis and remodeling. For skin healing and ulceration, treatments such as negative pressure bandages or ultrasound have been used to treat impaired tissue regeneration, but the efficacy of such treatments to stimulate angiogenesis and remodeling is debated.

Therefore, there is a need for an intentional agent (device and/or method) to promote healing, such as to stimulate angiogenesis and tissue remodeling, in order to aid in tissue and cellular healing. Such an intentional device may include vibration and/or stimulation, along with thermal actions, and optionally, have a shape suitable to conform to the body. Such a device optionally would be reusable, easy to clean, and optionally be usable with or without outside power sources, such as for use in the developing world (for example, operation with a power cord, with a battery, with a solar or light cell, or without the need for external power). As ease of use is helpful in this environment, embodiments that include rechargeable options, optional numbers of vibration units, or incorporation of thermal elements, cold or heat.

Accordingly, there is a need for a device and method that allows for the local improvement of biochemical and metabolic cellular and tissue mechanisms to improve healing of injured sites in a subject. There also is a need for a device and method using vibration or a combination of vibration and a thermal element that is applied to a subject to promote the healing of an injured site. It is to these needs and others that the present invention is directed.

### SUMMARY

The present invention comprises methods and devices for treating injured sites in a subject, such as for injury and wound healing and tissue repair, using devices capable of vibration and/or thermal effects. A method disclosed herein comprises contacting a device disclosed herein near, adjacent to or directly to an injured site of a subject, and initiating vibration and/or thermal effects, for a time sufficient to effect an increase in healing of the injured site.

A method disclosed herein comprises treating one or more injured sites in a subject, such as for treating an injured site or area by providing wound healing and/or tissue repair, comprising contacting a device disclosed herein on or adjacent to an injured site of a subject, such as a chronic wound, a burn, an acute or overuse injury, myofascial injury or inflammation; initiating vibration by the device in an intermittent or continuous vibration (which provides stimulation to the application site), optionally applying cold or heat simultaneously with the vibration, vibrating for a predetermined time, and optionally moving the device to another injured site or to a second location adjacent to the first injured site. For example, for an acute or overuse injury, a device may be applied directly to the injury site. For example, at sites wherein skin integrity is disrupted, a method may comprise placing a device adjacent to the disrupted site. At sites where the skin integrity is not disrupted, a method may comprise placing a device directly on the site.

A method disclosed herein comprises accelerating healing of one or more injured sites in a subject, such as for wound healing and tissue repair, comprising contacting a device disclosed herein on or adjacent to an injured site of a subject, such as an acute or chronic injury or chronic wound or a burn; initiating vibration by the device in an intermittent or continuous vibration, optionally applying cold or heat simultaneously with the vibration, vibrating for a predetermined time, and optionally moving the device to another injured site or to another location adjacent to the first injured site.

A method disclosed herein comprises treating a diabetic foot ulcer, comprising, contacting a device disclosed herein at or adjacent to a site of diabetic ulcer, initiating vibration by the device, optionally providing a thermal effect simultaneously with the vibration. The method comprises providing vibration and/or thermal treatment to the site of the ulcer for multiple times and such treatment modulates the condition of the ulcer, for example, by aiding in skin closure and eventual resolution of the ulcer, and/or relieving the pain caused by the ulcer.

Methods disclosed herein for treating injured sites by enhancing wound healing or repair at injured sites are also effective in reducing pain at such sites. For example, by enhancing wound healing, there is generally a reduction in pain at that site. For example, by treating myofascial injury, myofascial pain is reduced. Methods of enhancing wound or tissue healing also comprise methods for reducing pain at an injured site. Methods of the present disclosure comprise methods for reducing pain associated with wounds or injured sites by applying a device disclosed herein near, adjacent to or on a wound or injured site, initiating thermal and/or vibratory effects to relieve pain associated with the wound or injured site.

A method disclosed herein comprises treating an acute injury or an overuse injury, comprising, contacting a device disclosed herein at or adjacent to a site of injury, initiating vibration by the device, optionally providing a thermal effect simultaneously with the vibration. The method comprises providing vibration and/or thermal treatment to the site of the injury for multiple times and such treatment modulates the condition of the injury, for example, by aiding in reduction of swelling and eventual resolution of the injury, and/or relieving the pain caused by the injury.

A device disclosed herein comprises a casing, which may be shaped to conform to the contour of a surface. Such a shaped casing may have an established shape, for example, by a preformed casing, or may be a moldable casing that is shaped by a user. A casing may contain a vibratory element, and optionally a thermal element may be associated with the casing. A device disclosed herein comprises a casing, which may be shaped to conform to the contour of a surface that contains a vibratory element, and optionally a thermal element. In an aspect, a casing or at least one surface of a casing, is shaped to fit a curved surface of the body. For example, one surface of a casing may be concave, shaped like the inner surface of a circle, and when the device contacts a surface, such as a foot, the concave surface of the casing substantially contacts the foot surface, meaning that a majority of the concave surface is in contact with the area of the surface. The contact of the concave surface of the device allows for enhanced transfer of vibration and/or thermal effect to the surface. In an aspect, a casing or at least one surface of a casing is shaped to fit a curved surface of the body. For example, one surface of a casing may be convex, shaped like the outer surface of a circle, and when the device contacts a surface, the convex surface of the casing substantially contacts the surface, meaning that a majority of the convex surface is in contact with the area of the surface. This contact of substantially the entire convex surface of the device allows for enhanced transfer of vibration and/or thermal effect to the surface.

Vibration effects can be provided by any of the known vibratory devices such as, for illustrative purposes, a vibratory motor or an eccentric flywheel motor provided within the casing. Once vibration is initiated by providing power to the vibratory source, such as a vibratory motor, the vibration may be constant and continual, or the vibration may be intermittent, and cycle on and off at the same or a different vibration speed or frequency. Though not wishing to be bound by any particular theory, it is believed that intermittent vibration may aid in reducing or preventing habituation by the body to the vibrations. Alternatively, constant vibration during a treatment period may provide benefits to healing. One of skill in the art is able to determine which treatment method, constant or intermittent vibration, is suitable for a particular subject or ailment to be treated.

An exemplary embodiment of the device comprises a casing or moldable shape housing the various components of the invention and an optional strap or thermal pouch (e.g. neoprene) for holding the device to the subject. The casing may be manufactured of a stiff material to transmit vibration, and may be placed into a more flexible or pliant material in the form of a covering. The casing can be any shape or material able to be shaped, and preferably conforms to most body parts, particularly arms, shoulders and legs, or lower back or arch of foot. For example, an application area may be concave or convex so conform to rounded areas of the body to which the device may be applied. Any other shape may be employed, so long as the shape is large enough and structured so as to be able to contain the various working components as disclosed herein. A device disclosed herein may optionally comprise an adhesive area on a portion or substantially all of the proximal side of an application area or a thermal element for affixing the proximal side of the casing/thermal element to a surface, such as the body or skin of a subject.

A method disclosed herein comprises providing a device disclosed herein externally to the skin surface of a subject. For example, the subject may be a human or animal. The vibratory device disclosed herein may be placed at one or more injured sites in a subject, such as for wound healing and tissue repair, or may be placed proximal to such sites. In some methods, the vibratory device is placed at one site for a period of time and then moved to a second site. Methods disclosed herein allow for increased healing of an injured site, for example created by a burn or an injury, increased angiogenesis at an injured site, and reduction of pain.

These features, and other features and advantages disclosed herein will be apparent to those of ordinary skill in the relevant art when the following detailed description of the preferred embodiments is read in conjunction with the appended drawings in which like reference numerals represent like components throughout the several views. The figures and the detailed description which follow more particularly exemplify these and other embodiments of the invention.

### BRIEF DESCRIPTION OF THE FIGURES

The accompanying Figures, which are incorporated in and constitute a part of this specification, illustrate several aspects and together with the description serve to explain the principles of the invention.

FIG. 1 is a perspective view of an embodiment disclosed herein. In the view, the device (10) is being applied to the arm (100) of a subject with an injured site (104), which may be caused by an injury or burn. The positioning of the device (10) on the subject is adjacent to the injured site (104). Thus, for the illustrated injury site (104) on a subject's arm (100), the device (10) is placed adjacent to the injured site, for example, the device 10 may be placed approximately 0.5 cm to 15 cm from the injured site (104).

FIG. 2 is a top view of an embodiment of the invention with both vibration and cold capabilities. A minimal embodiment of the external features of the invention comprises the casing (12) and an on/off switch (16). Optional strap (14) can be used to hold the device on to the subject. Strap 14 can be attached to the casing (12) in any conventional manner or it can be an extension of casing (12) itself or it can be a continuous strap attached on both ends to the casing, such as a flexible band. The ends of strap (14) may have some type of connecting device (18), such as a hook and loop fastener, a clasp, a clip, snaps, magnets, adhesive, or the like for attaching the device about the subject's body part. Alternatively, if the strap (14) is flexible, the ends can be tied together around the subject's body part. The casing (12) of a device may be curved in a manner to enhance the proximity of the device by a strap or wrap.

FIG. 3 is a bottom view of an embodiment with both vibration and thermal capabilities. The casing (12) has a peripheral bottom rim (20) that defines an application area generally designated (22). All or a portion of application area (22) may contact the subject or may contact a thermal element thereinbetween the application area (22) and the subject. The application area (22) comprises vibration area (26) and/or a thermal area (24). Although thermal area (24) and vibration area (26) area shown as discrete areas, this is for illustrative purposes only, as there need not be any physical delineation between thermal area (24), vibration area (26), and application area (22) and these areas can overlap and be coexistent as the same area, which may be referred to as application area (22). Thermal element (28) cooperates with thermal area (24) to apply cold or heat to the subject, and vibrational source (32) (not shown) cooperates with vibration area (26) to apply vibration to the subject. Thermal area (24) and vibrational area (26) can occupy the same area, and form application area (22). Thermal element (28) may be located within thermal element pocket (34).

FIG. 4 is sectional side view of the embodiment as shown along the latitudinal line 4-4′ of FIG. 2. The casing (12) may be a generally hollow structure sized to contain a thermal element (28) and a vibrational source (32). Thermal element (28) can be placed within thermal element pocket through mouth or opening (36) and can be held within thermal element pocket (34) by friction, adhesives, fasteners, or by a zipper or other type of closure on mouth or opening (36). It is preferable that the bottom wall (38) of thermal element pocket (34) be sufficiently thin or having sufficient thermal transfer characteristics so as to allow the efficient transfer of cold or heat from thermal element (28) to the subject. The vibrational source (32) further comprises a power source (40) and wiring (42) electrically connecting vibrational source (32) and power source (40) to on/off switch (16).

FIG. 5A is a circuit diagram of an embodiment disclosed herein. A representative circuit diagram for the vibrational source (32) is shown. The vibrational source (32), power source (40), and on/off switch (16) are electrically connected in series by wiring (42). FIG. 5B is a circuit diagram of an embodiment disclosed herein comprising a vibrational source (32), a push button on/off switch (16), a battery power source (40), a control board (45), and optionally a speaker (46) and a light (47), in electrical connection in series by wiring (42).

FIG. 6 is a perspective view of an embodiment disclosed herein having an ornamental cover (60) or a decorated casing (12). The device is depicted in the same use as FIG. 1 with the addition of an ornamental cover (60) or a decorated casing (12).

FIG. 7 is a drawing of an exemplary device disclosed herein showing the application area (22) of a device (10) comprising the proximal side (the side closest to the subject when in use) (30) of the casing (12) and the proximal side (29) of a (transparent) thermal element (28) coextensive with the application area wherein thermal element (28) associated with the proximal side of casing (12) by use of an attachment element (50), which is an elastic band in this figure.

FIG. 8 is a drawing of an exemplary vibratory device disclosed herein wherein the device is placed on the injured site.

FIG. 9 A-E shows a drawing of an exemplary device disclosed herein having a concave shaped casing. FIG. 9A shows the front or anterior end (31) of the device (10) and its power switch (16), FIG. 9B shows the posterior or rear end (33) of the device (10) with its site indicator (52), and FIG. 9C shows the back or proximal side (30) of the device, that is contacts, or is placed proximally or adjacent to, the surface, having a clip (50) for holding a thermal element (not shown) in place; FIG. 9D shows a front or distal side (37) of the device (10), and FIG. 9E shows a side view (35a or 35b) of the vibratory device (10) where the attachment element (50) a clip, slightly protrudes from the posterior (lower) (33) proximal end and the on/off switch element (16) is shown at the anterior (upper) end (31).

### DETAILED DESCRIPTION

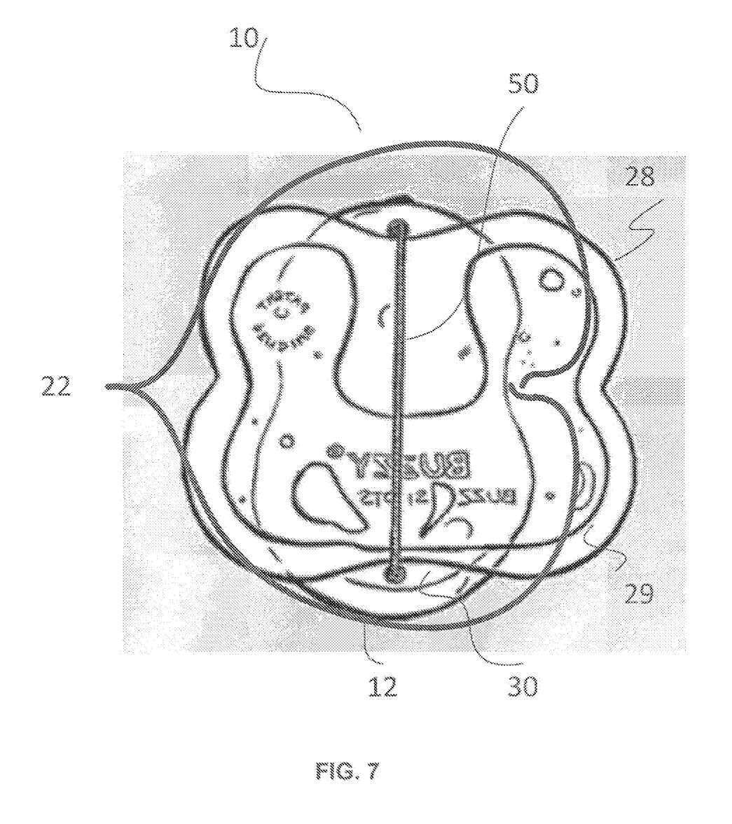

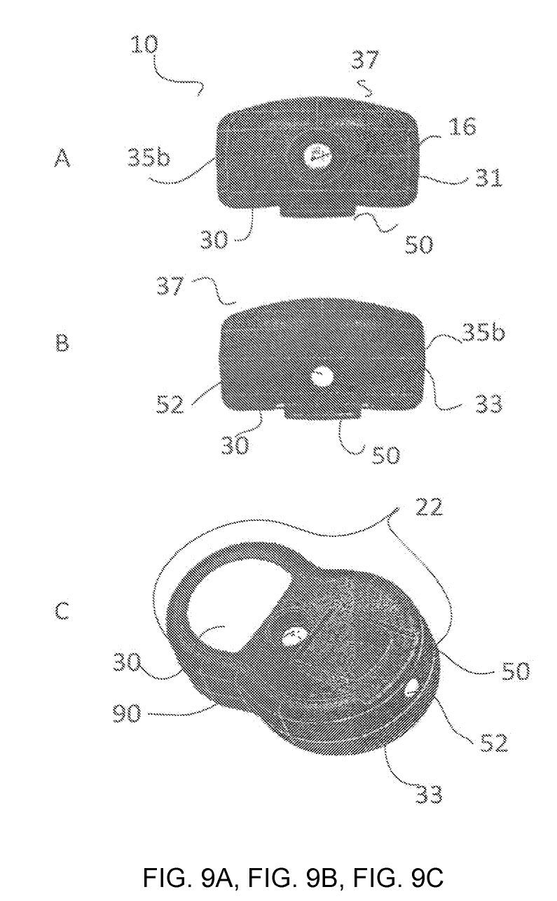

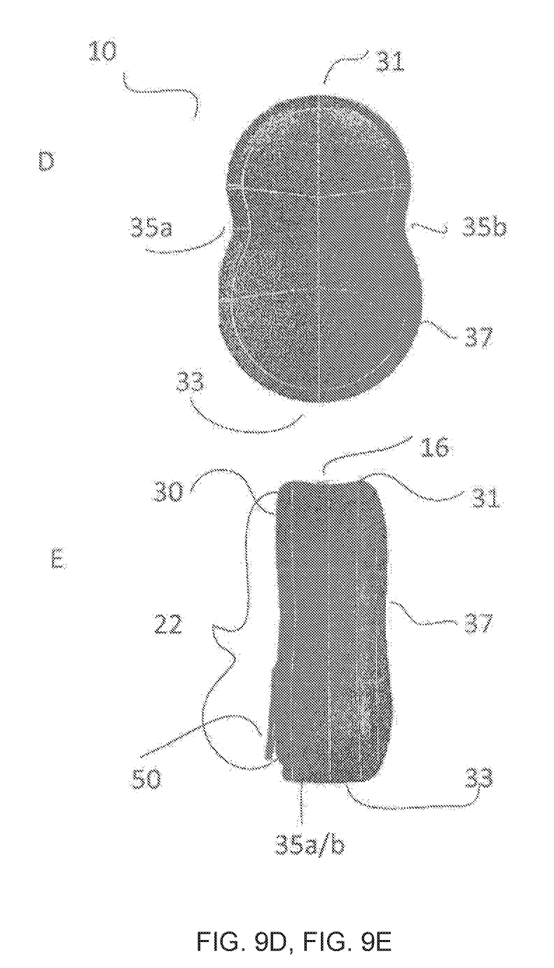

Disclosed herein are devices and methods for treating one or more injured sites in a subject, such as for wound healing and tissue repair. An embodiment of a device disclosed herein comprises a casing that contains a vibrational source, and an on/off switch for the vibrational source. A device may further comprise an attachment element for holding a thermal element in association with the casing. An attachment element (50) may be an integral portion of the casing, such as the clip shown in FIG. 9, or may be itself attached to the casing, such as the attachment element (50) shown in FIG. 7. An optional strap or wrap can be used to hold the device on to the subject, for example a strap that extends around a limb of the subject to secure the device on the limb of the subject such that the medical practitioner need not have an assistant present to hold the device, so that the medical practitioner can have both hands free to treat the subject, or a larger wrap that secures the vibratory device to a limb or body structure, for example, for an extended time. Further, a strap can act as a tourniquet, if necessary. Alternatively, the device can be held against the subject by the practitioner, the practitioner's assistant, or the subject.

A casing of a device disclosed herein comprises an application area that comprises a vibrational area and optionally, a thermal area. The application area is the portion of the casing for contacting the surface or for contacting a thermal element that in turn contacts the surface. For simplicity of understanding, the surface may be the skin of a subject. In an aspect, the application area may be all, or a portion, of the proximal side of a vibratory device. A thermal element cooperates with the thermal area to apply cold or heat to the subject, and a vibrational source cooperates with the vibrational area to apply vibration to the subject. As used herein, thermal element is intended to provide heat or cold, and one of skill can determine whether the method includes application of heat or cold. The placement of the thermal element on the device is variable so long as the effects of the thermal element can be felt on the subject so as to produce thermal effects (heat or cold) to the surface. The placement of the vibrational source in the casing of the device is variable so long as the vibrational effects of the vibrational source can be felt on the subject so as to produce vibrational effects to the subject. The walls of the casing define an interior space that is sized to contain at least the vibrational source, and a power source, such as batteries, and optionally a control element, a thermal element, a sound element, or a light element, and wiring to connect at least the vibrational source and the power source. An attachment element, a clip or hook on the proximal side (30) of the device, facing the subject surface, may be used to secure a thermal element to the exterior of the device. An attachment element, such as an elastic band may also be used to secure the thermal element to the proximal side of a device disclosed herein. Adhesive on the proximal side of the casing may be used to hold a thermal element to a device. A casing may further contain a control element for controlling the speed of vibration or period of vibration, for storing and providing sound, for providing a timing element, for controlling a light, such as turning a light on or off, with or without a timer, or making the light blink at a particular time point

Devices and decorative casings, and the use of distractive media are disclosed in U.S. Pat. No. 8,147,533 filed Oct. 4, 2006, PCT Application and issued Apr. 3, 2012, U.S. patent application Ser. No. 11/867,630, filed Oct. 4, 2007; Ser. No. 12/371,206, filed Feb. 13, 2009; and Ser. No. 13/426,330, filed Mar. 21, 2012; Ser. No. 13/795,683, filed Mar. 12, 2013; Ser. No. 14/635,540, filed Jan. 1, 2014; each of which is herein incorporated in its entirety.

A casing may further comprise on opening through a wall of the casing for providing an amplifier on the outer surface of the casing that is connected to a control element or a sound element contained within the casing. A casing may further comprise on opening through the casing for providing a light, such as an LED light, on the outer surface of the casing that is connected to a control element or a timing element contained within the casing. A light (and/or sound) may be turned on when vibration is initiated and turned off when power to the vibration element is turned off. Alternatively, powering on the vibration element may also power on a timing element, and optionally a light (and/or sound), so that when a desired time period has occurred, the timing element may turn off the light (and/or sound), or may turn off a light (and/or sound) and the vibration element, or the timing or control element may turn on sound or light after a period of vibration. Alternatively, the timing element may be under a control that is separate from a control for the vibration element. Components, control elements, such as a polycarbonate circuit board and the programming to accomplish the disclosed activities and others, and elements such as timing elements, sound elements and lights, are known, and can be selected or commercially acquired by those of skill in the art. Wires for connecting the elements within the casing or on the surface are contemplated by the present invention.

A casing may be shaped to provide an application area that is in contact with a surface so that substantially all of the application area contacts the surface. For example, a casing may be flat or concave in shape on at least its proximal side so that the application area is shaped so that substantially all of the proximal side of the casing contacts the surface of the surface. When a thermal element is placed on the proximal surface of the casing, substantially all of the proximal surface of the casing contacts the thermal element interposed between the casing outer surface and the contacted surface so that an area of the surface that is equivalent to the area of substantially all of the proximal side of the casing is contacted by the thermal element and receives vibrational effects therethrough (the application area). All or a portion of a casing may be curved. For example, the entire casing may be curved, such as in a concave direction (curved like the interior of a circle), so that the proximal side of the casing is contacting a surface through all or a portion of its surface (or the thermal element interposed therebetween) and the distal side (37) of the casing is curved to mirror the curve of the proximal side, so as to be comfortably held by a hand or held in place by a strap. Alternatively, only one surface, either the distal or proximal side may be curved, for example, where the proximal side is flat, but the distal side is curved. Additionally, a proximal side and/or a distal side of the casing may be flat or planar, and the lateral sides (35a or 35b) of the casing may be shaped, for example, as shown in FIGS. 7 and 9, there may be an indented area in the lateral sides. The lateral sides of the casing may be shaped in any form desired.

A casing may further comprise an adhesive area on a portion or substantially all of the application area. In an aspect, an adhesive area may be found on the proximal side of a thermal element and may cover a portion or substantially all of an area on the proximal side of the thermal element that corresponds to the application area of a device. A device disclosed herein may optionally comprise an adhesive area on a portion or substantially all of the proximal side of the application area of the casing or the thermal element for attaching the proximal side of the device/thermal element to a surface, such as the body or skin of a subject. On the casing and/or thermal element and before use, the adhesive area may be covered by a removable shield. In use, the removable shield is removed from the adhesive area on the proximal side of the casing and/or thermal element so that the adhesive is exposed and is capable of affixing the proximal side of a casing to a thermal element or to a surface, such as skin, or is capable of affixing the proximal side of a thermal element to a surface. Optionally, a surface of the casing or thermal element other than the proximal side of the device may have an adhesive area on a portion or substantially all of the surface of the device other than the proximal surface. The adhesive area on such a surface may be covered by a removable shield. In use, the removable shield is removed from the adhesive area of the casing and/or thermal element so that the adhesive is exposed and is capable of affixing the side of a casing to a thermal element or to a surface, or is capable of affixing the side of a thermal element to a surface. A device of the present invention may have one or more adhesive areas on a casing and/or on a thermal element. For example, a device of the present invention may have one adhesive area on a casing and one adhesive area on a thermal element. For example, a device of the present invention may have two adhesive areas on a casing. For example, a device of the present invention may have two adhesive area on a thermal element. For example, a device of the present invention may have one adhesive area on a casing and two adhesive areas on a thermal element. For example, a device of the present invention may have two adhesive areas on a casing and two adhesive areas on a thermal element. Alternative arrangements of adhesive areas on one or more components of the disclosed devices are contemplated by the present invention.

The vibrational source can be any conventional vibrational source or means for producing high frequency low amplitude vibrations. The on/off switch can be a common switch or a push button on/off switch, and is used to turn the vibrational source on and off. Additionally, the vibrational source may be activated, not by a switch, but by the removal of an impeding material, that when the impeding material is removed, a circuit is completed and the vibrational source is activated. A power supply may be activated by removal of a tab such that removal of the tab allows for the completion of a circuit without the need for an on-off switch. Once turned on, the vibrational source may vibrate in a constant and continuous mode, or the vibrations may be noncontinuous, such as intermittent periods or cycles of vibration and no vibration. The power source for operating the vibrational source can be any type of power source such as but not limited to a connection to an alternating current source (a wall plug), a solar or other light cell, a miniature reactor, a mechanical source such as a flywheel or springs, a disposable or rechargeable battery or the like.

In a method, a device disclosed herein improves healing of an injured site that is contacted by the device, or wherein the device is in very close proximity to the injured site, through the use of vibrational and/or thermal modulation of cellular and biochemical mechanisms such as angiogenesis, reduction of inflammatory cells, lowering the level of inflammatory cytokines, and increasing the level of pro-angiogenic factors and growth factors. In response to contacting the skin surface with a vibrating and/or thermal device disclosed herein, healing of the injured site is improved. More specifically, disclosed herein is use of a device disclosed herein for concurrently applying a combination of vibrational effect and thermal effect to a surface area of a subject. In an aspect, the combined effects of vibration and thermal effects enhance healing and provide treatment to an injured site proximal to the site of application of a device.

For example, the effects of vibration and/or thermal application may provide a local physiological effect to a surface and its underlying ducts or vessels within from 0.0 cm to 15 cm from the site of application of the device. Application of the device may have a more systemic effect by triggering a response in the local area, (0.0 cm to 15 cm) by a body part that has effects at a distant location, such as triggering cellular changes at the site of application of the device that causes attraction of cells or growth of blood vessels in the injured area. It is known that vibration helps to reduce pain as the vibrational, or motion, nerves surmount the pain nerves, which is known as gate theory to those of ordinary skill in the field. Similarly, it is known that cold helps to reduce pain as the temperature nerves surmount the pain nerves. It also is known that warm thermal contact is effective at vasodilation. Stimulation of aDelta nerves, which is accomplished by a device disclosed herein, may aid pain reduction, which may relax tense muscles.

Disclosed herein are methods comprising use of a device disclosed herein for aiding in, enhancing or increasing healing of an injured site. The present invention comprises methods comprising use of a device disclosed herein for healing, such as, but not limited to, increasing biochemical and cellular responses that lead to healing of an injured site, such as increased granulation or skin production at a chronic wound or burn. A method disclosed herein comprises contacting a device disclosed herein to a site of injury in a subject, for example, to a chronic wound or burn, initiating vibration and/or thermal effects, for a predetermined time, such as a time sufficient to effect modulation of cellular and biochemical mechanisms such as angiogenesis, reduction of inflammatory cells, lowering the level of inflammatory cytokines, and increasing the level of pro-angiogenic factors and growth factors. For example, a device disclosed herein is provided to an area of the subject where the skin is injured, such as a burn or a chronic wound. For example, the device, comprising a thermal element, is placed on a chronic wound and is held in place. The device's switch is activated so as to turn on the vibration element, and the thermal and vibrational effects are transmitted to the acute or chronic injury or wound area. This treatment may be repeated hourly or daily or for any length of time to modulate cellular and biochemical mechanisms such as angiogenesis, reduction of inflammatory cells, lowering the level of inflammatory cytokines, and increasing the level of pro-angiogenic factors and growth factors. The treatment may continue for several minutes at a time, wherein the vibration and thermal effects are provided by the device to the chronic wound, and such treatments may be repeated one or more times a day for one or more days, weeks, or months. With repeated treatment, the extent of the injury is reduced and the injured site may be resolved. With reduction in injury, pain is also reduced. For example, treatment times may comprise from about 0.5 minutes to about 1 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 20 minutes to about 30 minutes, and ranges therebetween. The treatment times listed here are not contemplated to be limiting to the methods of treatment of the invention. One of skill in the art can determine optimal treatment time periods.

An injured site, such as an open wound in the skin or underlying structures, may be treated by medical personnel by scraping the wound with a blunt instrument to remove cellular debris or other debris present and this treatment is very painful, though necessary. For example, before or after such a treatment, the wound may be contacted by a device disclosed herein to treat the injured site and aid in healing the injured site. A method of treatment of an injured site comprises a) contacting a device disclosed herein with an area on the surface of a subject proximate to or on the site being treated by scraping, so that at least a portion of the application area of the device contacts the area; b) initiating vibration by the device in an intermittent or continuous vibration, and optionally applying a thermal effect simultaneously with the vibration, by interposing a thermal element between the application area of the device and the contacted surface; and c) continuing vibration and/or thermal effect at the site of for a predetermined time. The thermal effect may cold or warm. The device may be placed on one or more portions of the injured site or from 0.5 cm to 15 cm from the injured site.

A burn site in the skin or underlying structures may be treated by medical personnel and burn treatment may be very painful, and the healing period after a burn may be very painful or pruritic (itchy) even if no active treatment is made to the burn area. A method of treating a burn or relieving pain or itching while healing comprises a) contacting a device disclosed herein with an area on the surface of a subject proximate to or on the site being treated, so that at least a portion of the application area of the device contacts the area; b) initiating vibration by the device in an intermittent or continuous vibration, and optionally applying a thermal effect simultaneously with the vibration, by interposing a thermal element between the application area of the device and the contacted surface; and c) continuing vibration and/or thermal effect at the site of for a predetermined time. The device may be placed on one or more portions of the injured site or from 0.5 to 15 cm from the injured site.

Methods of use of a device disclosed herein include, but are not limited to, methods to control pain associated with injections, venipuncture, IV starts, cosmetic injections (e.g., Botox injections), temporary relief of minor injuries (muscle or tendon aches, splinters and bee stings), and treatment of myofascial pain caused by trigger points, restricted motion and muscle tension. Wound healing and pain relief treatment methods are also accomplished by use of a device disclosed herein to provide thermal and/or vibratory effects to one or more sites on the subject.

A thermal element is cooled or heated, if necessary to within a predetermined temperature range. The thermal element may be placed within the casing or attached to the casing. Alternatively, if the device is made to certain standards, the entire device already containing a thermal element can be cooled to the desired temperature. When a subject anticipates a need for a treatment using the device, the device is applied to the body at a desired location, such as at the site to be treated or at a site proximate to an injured site, as described for treatments disclosed herein.

When an injured site is to be treated, a device disclosed herein comprising a thermal element may be applied to a selected area of the subject such that the vibrational area contacts the subject's skin through the thermal element to provide vibrational and thermal effects to the subject. The optional thermal element may be allowed to act upon the subject for a time necessary to initiate thermal effects, which can be for a period of seconds up to a period of several minutes or hours. For example, thermal treatment times may comprise from about 0.5 minutes to about 1 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 20 minutes to about 30 minutes, and ranges therebetween. The treatment times listed here are not contemplated to be limiting to the methods of treatment of the invention. One of skill in the art can determine optimal thermal treatment time periods. Once suitable thermal effects are achieved, or concurrently when the thermal element is applied to the subject, the vibrational source is actuated by the on/off switch, creating vibration. The vibrational source is allowed to act upon the subject for a time necessary to initiate vibrational effects which can be for a period of seconds up to a period of several minutes or more, or may be from about 0 to about 60 seconds. If prolonged vibratory and/or thermal treatment is desired the device may be applied for a longer period. The device may be removed from the subject, and/or the thermal element can be removed from acting on the subject and/or the vibrational source can be turned off. However, it is possible to leave the device, including the active thermal element and the active vibrational source in contact with the subject for prolonged periods of time. For example, the device may be left in place by using a wrap, and the device is then activated on an on-going schedule of time periods of use of the device and quiescence. One or more thermal elements may be provided to the device to allow for thermal effects to the subject during the periods of use.

“Thermal effects” as used herein includes, but is not limited to, the use or application of cold or reduced temperature (or the removal of heat) thermal elements or use of warm or heated thermal elements to a subject to induce a thermal effect in the subject.

“Vibrational effects” as used herein includes, but is not limited to, the use or application of vibration to a subject to induce vibrational responses in the subject. Vibrational effects also includes stimulation of the subject, in that vibration provides a stimulatory effect to contacted tissues or nerves, and may also stimulate adjacent or more distant sites of the subject.

“Vibrational and thermal effects” as used herein includes, but is not limited to, the use or application of either heat or cold or reduced temperature (or the removal of heat) concurrently, substantially concurrently, or sequentially with the use or application of vibration to a subject to induce physiological changes in the subject in the area contacted by a device disclosed herein or in a proximal or distal area.

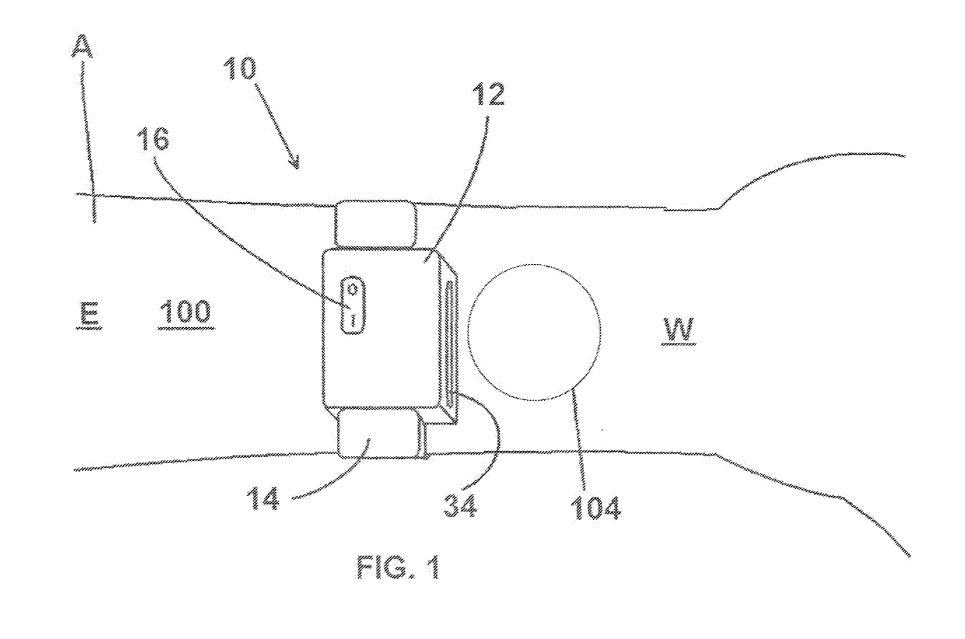

Referring now to FIG. 1, a perspective view of an embodiment of the device is shown as applied to the arm of a subject, showing the casing that houses the various components of the invention and an optional strap for holding the device to the subject In the view, the device (10) is being applied to the surface (100) of a subject who has an injured site (104). The positioning of the device (10) on the subject may be adjacent to the injured site or may be on the injured site. The on/off switch (16) is shown on the distal side of the device or casing, and a strap (14) is shown holding the device on the subject. A cuff, wrap, bandage or other similar component can be used in place of a strap to hold a device disclosed herein on a subject.

A casing (12) may be manufactured of a flexible or pliant material such as for illustrative purposes a natural or synthetic woven or non-woven fabric, a rubber or other flexible polymer material, a silicone-based material, or may be a rigid material, such as a plastic, metal or wooden casing, wherein the casing is a container with walls to define an enclosed area. Other flexible or pliant or other materials may be employed, and the material of construction may be non-toxic, hypo-allergenic and non-staining to the subject. A material that will transfer vibrations is contemplated by the present invention.

The casing can be any shape, and preferably is in the shape of a three-dimensional polygon (for use with an adult use) or an animal or other distractive shape (for use with a child) and the casing walls define a interior space or interior sections for containing the operating elements of the invention. Any other shape (as used herein, the term shape is used in the broad sense of three-dimensional works) may be employed, so long as the shape is large enough and structured so as to be able to contain the various working components of the invention as more fully disclosed below.

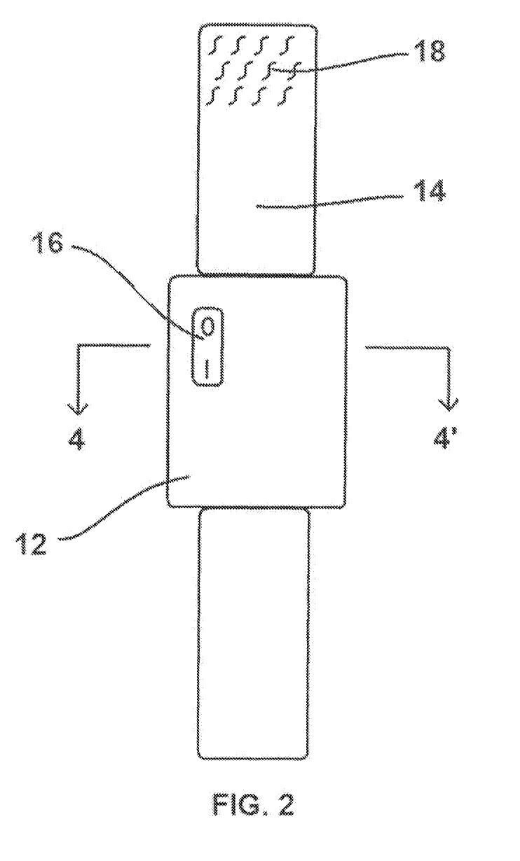

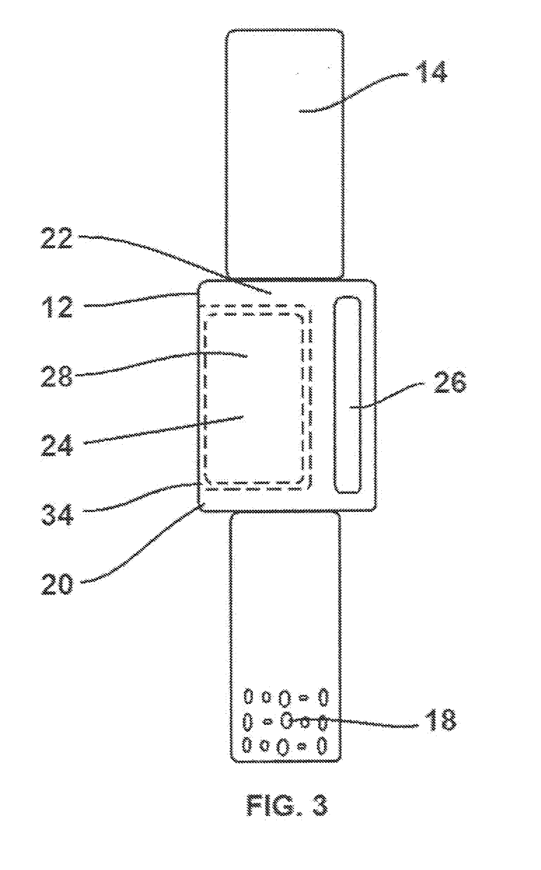

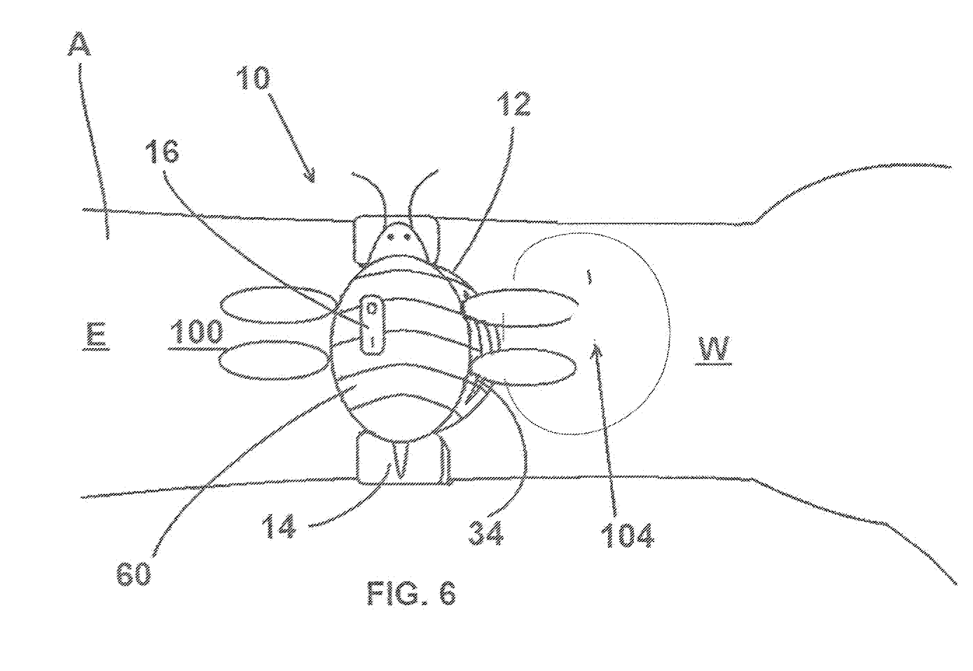

The device is shown in FIG. 1 applied adjacent to an injured site of a subject. A thermal element pocket (34) in the casing is illustrated on this embodiment. An embodiment showing the distal side of the invention of FIG. 1 is shown in FIG. 2, comprising the casing (12) and an on/off switch (16). An optional strap (14) can be used to hold the device on to the subject. Alternatively, the device can be held against the subject by medical personnel, or the subject. The strap (14) can be attached to the casing (12) in any conventional manner or can be an extension of casing itself. For example, the strap and casing can be attached together much like a conventional watch and watchband with hinges or pins. Or in another embodiment, the strap can be an extension of the fabric or other material enclosing the casing, such as an extension of a decorative cover (shown in FIG. 6). The ends of strap preferably have some type of connection element (18), such as a hook and loop fastener, adhesive, a clasp, a clip, snaps, magnets, or the like for attaching the device about the subject's body part. Alternatively, if the ends of the strap are flexible, the ends can be tied together around the subject's body part. Alternatively, the strap can be a continuous band, with both ends attached to the casing or cover.

Referring now to FIG. 3, a bottom or proximal view of an embodiment of the invention of FIG. 1 is shown. The casing (12) has a peripheral bottom rim that defines an application area (22). Application area (22) comprises thermal area (24) and vibration area (26). Although thermal area (24) and vibration area (26) area shown as discrete areas, this is for illustrative purposes only, as there need not be any physical delineation between thermal area (24), vibration area (26), and application area (22) and these areas can overlap and be coexistent as the same area, which may be referred to as application area (22). Thermal element (28) cooperates with thermal area (24) to apply cold or heat to the subject, and vibrational source (32) (not shown) cooperates with vibration area (26) to apply vibration to the subject. Thermal area (24) and vibrational area (26) can occupy the same area, or can coextend and form application area (22). Thermal element (28) may be located within thermal element pocket (34). The thermal pocket (34) is a slot, fold or other type of compartment in the casing into which the thermal element can be placed. As shown in FIG. 3, the thermal element pocket is accessed on the side of the casing via a mouth or an opening in the casing. Alternatively, the opening for the pocket can be located at other sites on the casing depending on the size and shape of casing and the location of the vibrational source within the casing. Alternatively, the thermal element can be contained within the main housing volume of the casing. Thus, the placement of the thermal element is variable so long as the cooling or heating effects of the thermal element can be felt on the subject so as to produce thermal effects. Thermal area in its simplest form is an area of the application area on the device that allows the thermal effects from thermal element to contact the subject.

The vibration area is an area on the casing in vibratory contact with the vibrational source. As disclosed in more detail below, vibrational source preferably is contained within the main housing volume of the casing. The placement of the vibrational source is variable so long as the vibration effects of vibrational source can be felt on the subject so as to produce vibrational effects. As shown in FIG. 3, vibrational area is proximal to thermal area; however, vibrational area can coextend with thermal area. Vibrational area in its simplest form is an area on the application area on the device that allows the vibrations from vibrational source to contact the subject.

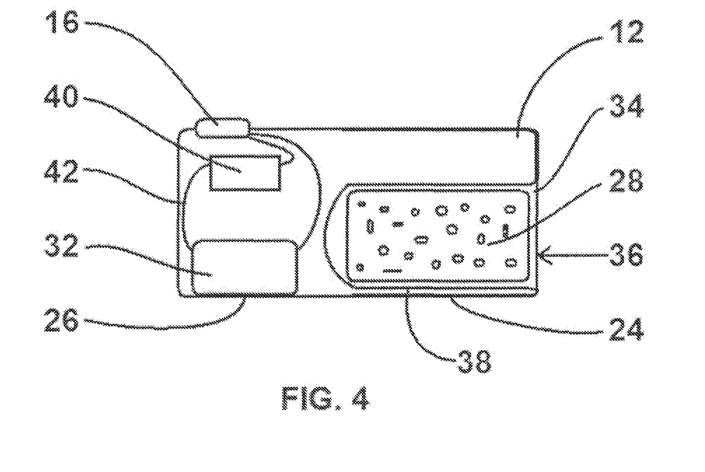

Referring now to FIG. 4, a sectional side view of the embodiment of the invention as shown along line 4-4′ of FIG. 2 is shown. The casing (12) is a generally hollow structure sized to contain an optional a thermal element, and a vibrational source. A casing (12) can be a rigid hollow container having an interior volume or a flexible or pliant container having an interior volume. Such containers are known, as well as their materials and methods of construction are within the skill of those in the art. The casing may be constructed such that casing (12) can contain and hold a vibrational source, and optionally, a thermal source, in a predetermined position relative to the subject when the device is contacting a subject.

As shown illustratively in FIG. 4, thermal element is contained in thermal element pocket. Thermal element can be placed within thermal element pocket through mouth or opening and can be held within thermal element pocket by friction, adhesives, fasteners, or by a zipper or other type of closure on the pocket mouth or opening. It may be desired that the bottom wall of thermal element pocket be sufficiently thin or have sufficient thermal transfer characteristics so as to allow the efficient transfer of cold or heat from thermal element to the subject. Alternatively, a thermal element (28) may be placed on the outside surface of the proximal side of a casing so that in use, the thermal element is interposed between the proximal surface of the casing and the subject's surface.

Thermal element can be any conventional thermal element capable of storing and transferring cold (removing heat). Illustrative examples of suitable thermal elements include metal ingots, low freezing point (below about 45° F. or 7.2° C.) liquids and gels, ceramics, polymers, polymer materials, natural materials such as bran, other heat sinks, hot packs, chemical reactive thermal packs, thermal gel packs, and even ice packs. Such thermal elements are known. It is only important that thermal element be able to transfer cold or heat to the subject in a sufficient amount so as to produce the desired effect, for example vasodilation, pain reduction, itching sensation reduction, or reduction in blocked vessels. For example, providing a temperature of below about 45° F. or 7.2° C., and between about 28° F. or −2.2° C. and about 54° F. or 12.2° C., or between about 38° F. or 3.3° C. and about 45° F. or 7.2° C., or for example about 34° F. to the subject prior to and during the treatment method is sufficient to provide a suitable level of effective thermal treatment. The thermal element is applied to the subject for a time period sufficient of treatment, and the application of the device may provide a thermal effect and/or a vibration to provide a suitable level of effective treatment by the device.

The thermal element may be any conventional thermal element capable of storing and transferring heat or cold. Illustrative examples of suitable thermal elements include high specific-heat capacity material like grains, such as wheat or buck wheat, sewn within an insulated fabric such as flannel, chemical thermal elements like calcium chloride- or supersaturated sodium acetate-based heat pads, or other conventional heat/cold packs. A thermal element may be a gel or other type of heat/cold pack that may be placed in a freezer or microwave and such heat/cold packs are known in the art. The present invention contemplates use of thermal elements that are known in the art. The thermal element needs to transfer heat or cold to the subject in a sufficient amount. One of skill in the art, such as medical personnel, or a subject, can determine an adequate temperature and time for application of the thermal element for methods disclosed herein. The thermal element is applied to the subject for a time period sufficient to cause the desired effect. A second or third thermal element may be used in replacing a first thermal element used in a method, especially in methods where application of vibration and/or thermal effects continue for a longer time period than the first thermal element can maintain the desired temperature.

As shown illustratively in FIG. 4, the vibrational source (32) is contained within the interior of the casing (12). Vibrational source (32) can be placed within casing (12) during manufacture or at any time after manufacture. An ingress and egress element (90) is preferred, as one embodiment of vibrational source utilizes a battery as the power source, and it may be necessary to change the battery on occasion. See FIG. 9C where an ingress/egress element (90), as illustrated, is a screw, is shown for opening the casing (12), and may be used for holding a control board, and/or power source and/or vibrational source (motor), or other components on a control board in the interior of the casing (12). Ingress/egress element (90) can be a snap, a screw, a bolt, or any closure components that would releasably hold the casing closed and allow for access to at least a power source within the casing. Vibrational source (32) and power source (40) can be held within casing by friction, adhesives, fasteners, or other types of securing means. Alternatively, the interior volume of casing can be approximately the same dimensions as the vibrational source, including the power source, such that additional means for securing the vibrational source 28 are unnecessary. In an aspects, the proximal side (30) of the casing which is adjacent to vibrational source be sufficiently thin or have sufficient vibrational transfer characteristics so as to allow the efficient transfer of vibration from vibrational source to the application area (22) of the casing (12) and thus to the subject to be treated in the methods disclosed herein.

Vibrational source (32) can be any conventional vibrational source or means for producing vibrations. As shown in FIG. 4, vibrational source further comprises a power source (40) and wiring electrically connecting vibrational source and power source to an on/off switch. Illustrative examples of suitable vibrational sources include elliptical flywheel motors, eccentric motors, and the like. Such vibrational sources are known. It is only important that the vibrational source be able to transfer vibration to the subject at a sufficient level to produce the effect intended in the disclosed methods. For example, a device disclosed herein can provide vibrations of between about 175-250 Hz. The application area of the device which vibrates due to the action of the vibrational source is applied to the subject for a time period sufficient to accomplish the effect intended in the disclosed methods, which can be between 0 seconds and several minutes or more depending on the subject and/or the method. For example, the application area of the casing may provide vibration to the subject for a period of about 0 seconds to about 60 seconds, or longer in certain methods, to accomplish the effect intended in the disclosed methods. A vibrational source may be a high frequency low amplitude eccentric motor. The motor may be controlled by a logic control board, such as a polycarbonate board, which known in the art. The motor and/or a power source may be held to a board by brackets, screws or other known attachment elements. For example, vibration treatment times may comprise from about 0.5 minutes to about 1 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 20 minutes to about 30 minutes, from about 1 to 2 hours, from about 1 to about 3 hours, from about 2 to about 4 hours, and ranges therebetween. The treatment times listed here are not contemplated to be limiting to the methods of treatment of the invention. One of skill in the art can determine optimal vibrational treatment time periods.

A vibrational source can produce a single vibrational cycle, multiple vibrational cycles, or be variable, for example in the vibrations per minute in a particular cycle, or in the number of vibrational cycles. In other words, the vibrational source can be a vibrational motor that operates at, for example, 4700 vibrations per minute or, for another example, at 5700 vibrations per minute, or in a range from about 6,000 to about 15,000 vibrations per minute, or from about 8,000 to about 14,000 vibrations per minute, or from about 9,000 to about 13,000 vibrations per minute, or any vibrations per minute thereinbetween. Alternatively, vibrational source can be a vibrational motor that operates at two or more vibrational cycles, for example, 9,000 vibrations per minute and 13,000 vibrations per minute, and can be switched between vibrational cycles by a switch or other control element. Alternatively, vibrational source can be a vibrational motor that operates at many different vibrational cycles along a continuum by using a potentiostatic switch, for example, vibrational source can be varied continuously or step-wise between 3000 vibrations per minute and 15,000 vibrations per minute. In an aspect, the vibrational source may provide intermittent vibration cycles, which may be the same or different vibrations per minute. For example, a vibrational source may provide 10,000 vibrations a minute for 4 seconds, stop vibrating for 4 seconds, thus completing one cycle of vibrations, then provide another cycle of vibrations at 10,000 vibrations a minute for 4 seconds, stop vibrating for 4 seconds, and so on. The vibrations per minute may remain the same for each cycle, or may vary randomly or vary in an increasing or decreasing manner. The time of vibration may vary randomly for each cycle, or may vary in an increasing or decreasing manner. The time of no vibration may vary randomly for each cycle, or may vary in an increasing or decreasing manner.

A switch may be a common switch and is used to turn the vibrational source on and off, namely to start and stop the vibration, respectively. The switch may also control power transmission to a control element or other element of the device, such as a sound element or a light. The switch can be secured to the casing at any convenient position where it may readily be actuated, or accessed remotely by wired or wireless components. As shown in FIG. 9, the switch is located at the anterior side (31) of the device and is a push button switch. The switch is electrically connected in a known manner between the power source and the vibrational source to control the application of power to the vibrational source. In an aspect, when the vibrational source is switched on, the vibrating force produced from the vibrational source, such as the various types of motors disclosed above, will be transmitted through the casing to the contacted surface.

A device disclosed herein may have more than one switch, each of which may control the power to an element of the device, or provide on/off control of the element itself, and discussion of one switch is not to be seen as limiting to the invention. A switch can be a common on/off switch, such as a toggle, lever, push-button, capacitance or other switch. For example, a device may be activated by the removal of a barrier so that a circuit is then completed. This completion of the circuit so that the device is activated is a type of switch. The disclosure herein contemplates a type of switch that would be practical with a single vibrational cycle motor. Alternatively, switch can be a common three-way switch. This type of switch would be practical with a double vibrational cycle motor. Alternatively, a switch can be a common potentiostat. This type of switch would be practical with a vibrational motor that operates at many different vibrational cycles along a continuum. The selection of the type of switch and the control element of a device is within the skill of those knowledgeable in the art. For example, a switch can turn power on or off to a control panel that in turn controls a vibration source, and/or other elements of the device, such as sound or light elements.

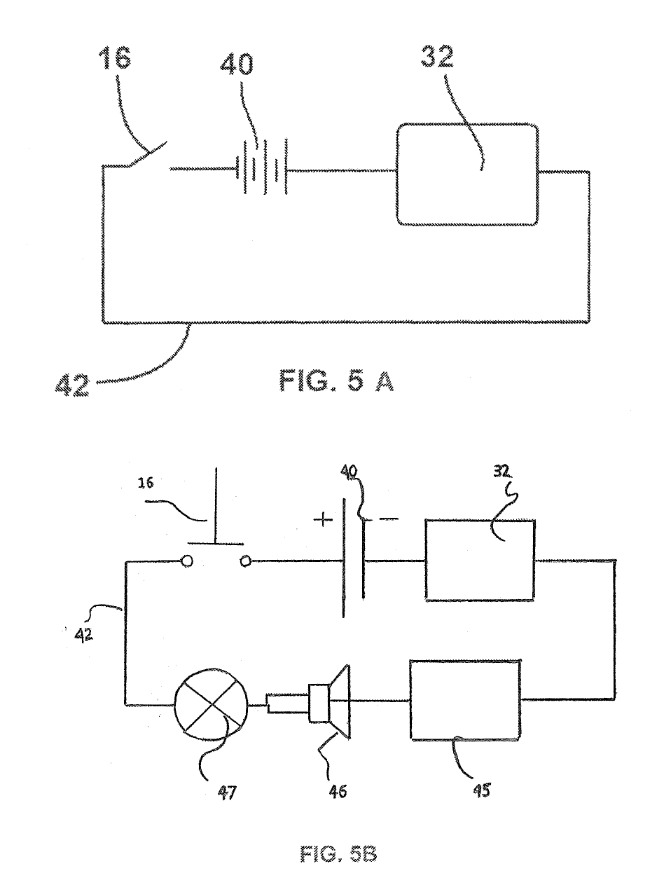

Referring now to FIG. 5A, a representative circuit diagram for the vibrational source is shown. Vibrational source, power source and on/off switch are electrically connected in series by wiring. Power source is illustrated in FIG. 5 as a battery; however, power source can be any type of power sources such as but not limited to a connection to an alternating current source (a wall plug), a solar or other light cell, a reactor, a mechanical source such as a flywheel or springs, or the like. It is only important that power source be able to provide sufficient power to vibrational source so as to produce sufficient vibration for effecting vibrational vasodilation. FIG. 5B is a circuit diagram of an embodiment disclosed herein comprising a vibrational source (32), a push button on/off switch (16), a battery power source (40), a control board (45), and optionally a speaker (46) and a light (47), in electrical connection in series by wiring (42).

A thermal element is cooled or heated, as necessary. For example, if the thermal element is a metal ingot or low freezing point gel, the thermal element is placed in a refrigerator, freezer, or other cold site. Alternatively, if the thermal element is a high specific-heat capacity material like a grain sewn within an insulated fabric it may be microwaved before use to heat it. When the thermal element is of a satisfactory temperature, the thermal element is placed within or adjacent to the casing. The thermal element may be placed within the thermal element pocket, within an attachment element (50) such as an elastic band attached to the casing so that the thermal element is interposed between the elastic band and the proximal side (30) of the device, or within an attachment element (50) such as a clip located on the proximal side of the device as shown in FIGS. 9 A-C and E. The device is contacted to the surface, such as the surface of skin of a subject, at the desired location, depending on the method employed for the desired treatment.

The application area of the device, optionally with the thermal element interposed therebetween, is applied to the selected area of the subject such that the application area, comprising the thermal area and the vibrational area, contact the subject's skin. The thermal element may be contacted with the surface for a time period, without vibration, for example, to allow the thermal element to act upon the subject for a suitable time period so as to initiate thermal effects. Alternatively, concurrently with application of the thermal element to achieve thermal effects, the vibrational source is actuated, for example, by pressing the switch, and starting the vibrational source, and vibrations are transferred through the application area (and through the thermal element if present) to the contacted surface. The vibrational source also is allowed to act upon the subject for a suitable time period so as to initiate the desired effect.

Once the desired treatment is completed, the entire device can be removed from contacting the surface, and/or only the thermal element can be removed and the device continues to provide vibration to the surface, or the thermal element may remain in place on the surface and the vibrational source may be turned off. In one illustrative method, the device is left in contact with the subject for a period of between 0 and 60 seconds, or for one or more minutes, and the treatment is repeated for one or more days.

Referring now to FIG. 6, alternative embodiments include casings having interesting or distracting shapes or ornamental covers (60) over the casing. For example, the casing could be a material in the shape of a bumble bee, as illustrated in FIG. 6. When the device is applied to a child, the distracting shape both can lessen the fear a child may have to device medical procedure.

Referring to FIG. 7, a proximal view of a device 10 disclosed herein comprising a thermal element 28 (shown as transparent so as to view the proximal side 30 of the device 10). An attachment element 50 (an elastic band) is shown holding the thermal element 28 to the application area 22 of the proximal side 30 of the casing 12 of the device 10.

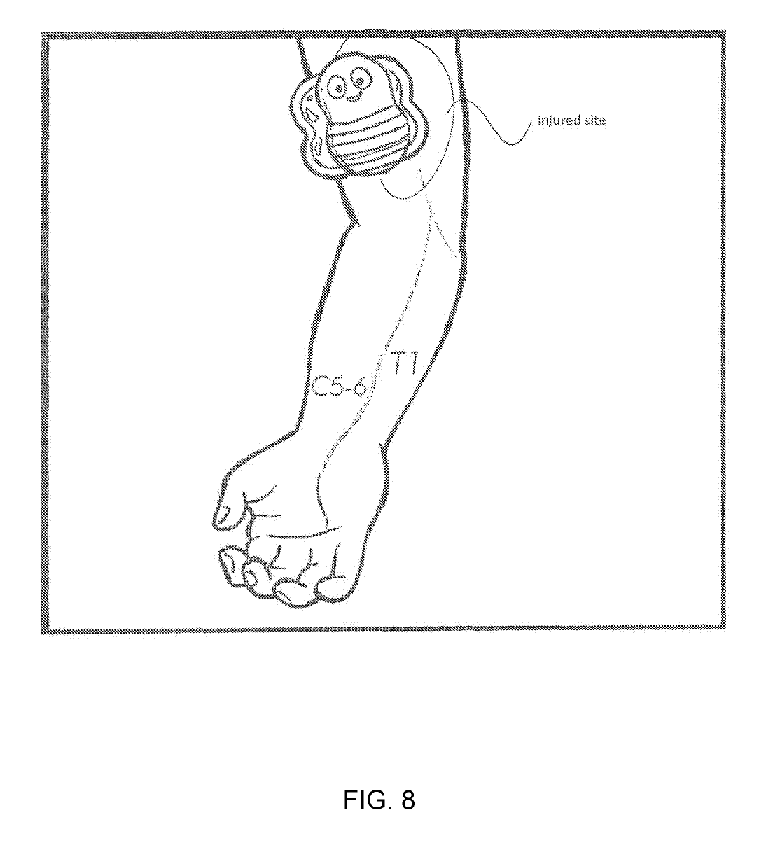

FIG. 8 shows the placement of a device disclosed herein 10 on the arm of a human.

FIG. 9 A-E shows a drawing of an exemplary device disclosed herein having a shaped casing. 9A shows the front or anterior end (31) of the device (10) and its power switch (16), B shows the posterior or rear end (33) of the device (10) with its site indicator (52), and 9C shows the back or proximal side (30) of the device, that is contacts, or is placed proximally or adjacent to, the surface, having a clip (50) for holding a thermal element (not shown) in place. The distal side (30) may be flat or planar in shape, or may be curved, as desired. D shows a front or distal side (37) of the device (10), and E shows a side view (35a or 35b) of the vibratory device (10) where the attachment element (50) a clip, slightly protrudes from the posterior (lower) (33) proximal end and the on/off switch element (16) is shown at the anterior (upper) end (31). The site indicator (52) provides a guide to the user for placing the device. In methods where applicable, the device is placed so that the indicator is directly at the injured site. 9C shows the proximal side 30 of the device 10. For example, the entire proximal side may be the application area 22 and the entire proximal side (30) substantially contacts the surface contacted and the proximal side (30) comprises the application area 22 through which vibration is transferred to the surface. In an aspect, only a portion of the proximal side may be the application area. Also shown is attachment element, 52, which is a clip. The indicator 50 is also shown. FIG. 9D shows the distal side 37 of the device which may be curved in a shaped manner to fit comfortably in a hand when gripped. The lateral sides 35 A and 35b are shown as indented in a generally midline position. Other shapes are contemplated by the invention and the indentions may or may not provide a functional or a decorative aspect to the invention. FIG. 9E shows a lateral side 35a/b view of the device 10. At the anterior end 31 the switch 16 is visible. The attachment element (clip) 50 is visible on the proximal side 30. The shape of the distal side (37) may be any desired shape, and may be in some embodiments flat or planar, or may be curved, either convex or concave in shape.

Thus, in one of its simplest forms, the invention is a device for providing vibration and/or thermal treatment to a surface, comprising a casing comprising an application area, wherein at least a portion of the application area is shaped to substantially contact a surface, such as a subject's skin, a vibrational source contained within the casing, with said vibrational source capable of producing vibration that is transfer through the casing to at least the surface, and optionally comprising a thermal element capable of transmitting heat or cold. The application area is constructed to allow the transmission of vibration from the vibrational source to the surface, such as a subject's skin, and by the interpositioning of a thermal element between the application area and the surface, providing thermal effects to the surface. The vibration or combination of the vibration and transmission of cold or heat from the thermal element produces vibrational and thermal effects on the subject.

Disclosed herein devices and methods of using a device further comprise the use of a removable thermal element. For example, the casing may comprise a flat hook on which a thermal element could be attached while still transmitting vibrational energy through the thermal pack to the subject.

Disclosed herein devices and methods of using a device further comprising a vibrational unit with a power source capable of being attached via an adhesive dressing (e.g. tegaderm) or attached to the skin in an array of vibrational units.

Disclosed herein is a kit comprising a vibratory device disclosed herein, a thermal element and instructions for use of the device.

As used herein, subject means a human or animal, and includes any living animal on the planet Earth.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

The above detailed description of the preferred embodiments, and the examples, are for illustrative purposes only and are not intended to limit the scope and spirit of the invention, and its equivalents, as defined by the claims. One skilled in the art will recognize that many variations can be made to the invention disclosed in this specification without departing from the scope and spirit of the invention.

### REFERENCES

Baxter A L, Leong T, Mathew B. External Thermomechanical Stimulation versus Vapocoolant for Adult Venipuncture Pain: Pilot Data on a Novel Device. Clin J Pain 2009; 25(8):705-710.
Baxter A L, Cohen L L, Lawson M L, Von Baeyer C L. A Randomized Clinical Trial of a Novel Vibrating Tourniquet to Decrease Pediatric Venipuncture Pain. Pediatr Emerg Care 2011; 27(12):1151-6.
Figueroa A, Gil R, Sanchez-Gonzalez M A. Whole-body vibration attenuates the increase in leg arterial stiffness and aortic systolic blood pressure during post-exercise muscle ischemia. Eur J Appl Physiol 2011; 111(7):1261-8.
Figueroa A, Gil R, Wong A, Hooshmand S, Park S Y, Vicil F, et al. Whole-body vibration training reduces arterial stiffness, blood pressure and sympathovagal balance in young overweight/obese women. Hypertens Res 2012.
Freeman A W, Johnson K O. A model accounting for effects of vibratory amplitude on responses of cutaneous mechanoreceptors in macaque monkey. J Physiol 1982; 323:43-64.
Hess H A. A biomedical device to improve pediatric vascular access success. Ped Nurs 2010; 36(5): 259-263.
Hollins M, McDermott K, Harper D. How does vibration reduce pain? Perception. 2014; 43(1):70-84.
Inal S, Kelleci M. Buzzy relieves pediatric venipuncture pain during blood specimen collection. MCN Am J Matern Child Nurs 2012; 37 (5); 339-45.
Imtiyaz S, Veqar Z, Shareef M Y. To Compare the Effect of Vibration Therapy and Massage in Prevention of Delayed Onset Muscle Soreness (DOMS). J Clin Diagn Res. 2014 January; 8(1):133-6.
Skoglund C R. Vasodilatation in human skin induced by low-amplitude high-frequency vibration. Clin Physiol 1989; 9(4):361-72.
Veqar Z, Imtiyaz S. Vibration Therapy in Management of Delayed Onset Muscle Soreness (DOMS). J Clin Diagn Res. 2014 June; 8(6):LE01-4.

### Examples

### Example 1 Increased Healing Using Vibration and Thermal Effects

Areas of pain or injury caused by overuse or muscle strain can be treated with a device, with or without thermal element, wherein the device is placed directly on the injury. For example, a rotator cuff injury was treated by placing the device with a thermal element touching the skin and the vibrational unit directly above the painful location. The vibration was activated, and is held or secured in place for 10-20 minutes two to three times per day. There was immediate pain relief during the treatment periods, and vibrational and thermal treatment continued through the time period that physical therapy was performed so that healing was enhanced and pain was reduced.

Wrist pain from inflammation and/or overuse was treated by attaching a cooled or warmed thermal element to the device, locating the thermal element so that it was touching the area where pain is felt or where pain was anticipated to happen prior to an activity, such as typing. The vibration was activated and the device is left in place 10-20 minutes. The activity, typing, was performed and the amount of pain perceived was lessened compared to typing without pretreatment with the device.

For knee strain treatment—he device with a cooled thermal element was placed directly on the area where a knee strain was painful. After placing the device, with the thermal element touching the skin, and vibration was activated. Once the vibration was activated, the unit was left in place 20-30 minutes two to three times per day. There was immediate relief during the virbratory/thermal treatment, and the knee pain was lessened and healing of the knee was enhanced after several treatments with the device.

For a burn in which itching occurred in the course of healing, the device with a thermal element was placed proximal, about 2-5 cm away from the injured area, with the cold pack (thermal element) touching the skin, and the vibration was activated. The device was left in place 10-20 minutes for relief as often as necessary to enhance healing of the burn and to relieve the pain of itching.

For treatment of an injured knee—During physical therapy where the knee is being flexed and extended, the device, with or without a thermal element, is placed adjacent to or on the area where pain was felt and the vibration was activated. The user (subject) attached the unit with a Velcro strap or held it in place for the duration of the physical therapy, The subject moved the device to a second location and continued the vibration and cooling to achieve maximum pain relief and stimulation of the affected knee.

For arthritic conditions—the device, and optionally with a warmed thermal element, was placed proximal to the area where pain was felt in the hip, so that the thermal effects are at the site of pain and the vibration was activated. The user attached the unit with a Velcro strap or held it in place for the duration of the vibrational/warm therapy. Immediate pain relief was felt, and with continued treatments, pain was lessened in the hip joint.

What is claimed is:

1. A method for increasing healing in a site of injury in a subject, comprising contacting a vibratory device at or adjacent to an injured site, initiating vibration by the device, optionally providing a thermal effect simultaneously with the vibration, and enhancing the healing in the site of injury.

2. The method of claim 1, wherein the injured site is a wound or burn.

3. A method for treating an injured site of a subject, comprising contacting a vibratory device disclosed herein at the injured site; providing vibration by the device in an intermittent or continuous vibration, optionally applying a thermal effect simultaneously with the vibration, and vibrating for a sufficient time to treat the injured site.

4. The method of claim 3, further comprising, after contact at the injured site, moving the vibratory device to a different site on or adjacent to the injured site, and initiating vibration at for a time sufficient to treat the injured site; and optionally applying a thermal effect simultaneously to the injured site.

5. The method of claim 3, wherein the injured site is a burn or a wound.

6. A kit comprising a vibratory device comprising a casing containing at least a vibratory motor and a power source; a thermal element, and instructions for use of the device and thermal element for treatment of a human or animal.
